(19) United States
(12) Patent Application Publication
(10) Pub. No.: US 2026/0347733 A1
(43) Pub. Date: Nov. 5, 2026

(54) METHOD FOR PRODUCTION OF BREWERS WORT

(71) Applicant: NOVOZYMES A/S, Bagsvaerd (DK)

(72) Inventors: Alexander MAUCH, Bagsvaerd (DK); Carsten ANDERSEN, Bagsvaerd (DK)

(73) Assignee: NOVOZYMES A/S, Bagsvaerd (DK)

(21) Appl. No.: 19/116,446

(22) PCT Filed: Oct. 12, 2022
(86) PCT No.: PCT/EP2022/078390

§ 371 Date: Mar. 28, 2025

(51) Int. Cl. C12C 7/047; C12C 5/00; C12N 9/26; C12N 9/34

(52) U.S. Cl. CPC C12C 7/047; C12C 5/004; C12N 9/2414; C12N 9/2428; C12Y 302/01001; C12Y 302/01003

(57) ABSTRACT

The present invention relates to a method for production of a brewer's wort using Sorghum in the grist.

### REFERENCE TO A SEQUENCE LISTING

This application contains a Sequence Listing in computer readable form. The computer readable form is incorporated herein by reference.

### FIELD OF INVENTION

The present invention relates to a method for production of a brewer's wort using Sorghum in the grist.

### BRIEF DESCRIPTION OF THE FIGURES

FIG. 1 shows an overview of the liquefaction process as run on the Rapid Visco Analyzer RVA 4500 (see Materials and Methods, Liquefaction assay). The top line is the Temperature curve, and indicates the temperature over time. The bottom line indicates the viscosity measurements taken over time. The performance indicators derived from in-situ viscosity measurements taken over the course of the liquefaction experiment are highlighted. The shaded area VA indicates the Viscosity Area (Area under Viscosity curve). The time span used for calculating the area is indicated by arrows. The measurement at point VBC is the viscosity before cooling. The final viscosity is indicated by FV and is measured at the very end of the process, after cooling. Set-back (SB) is measured as VBC (viscosity before cooling)−FV (final viscosity). (see Materials and Methods, Tables 3A; as well as Example 1 and Table 4).

FIG. 2 shows Final viscosities after the liquefaction process of Sorghum in presence of different alpha-amylases Y-axis Viscosity measured in cP (centipoise); x-axis for the different enzymes tested. (See Example 1 and Table 4).

FIG. 3 shows Area underneath viscosity curve from 10 min until end, derived from integration of the individual viscosity measurements during the liquefaction process of Sorghum in presence of different alpha-amylases tested in Example 2 (see Example 1 and Table 4).

FIG. 4 shows the Set-back values for the samples from Example 1 (see Example 1 and Table 4). X-axis: Enzymes tested; y-axis, viscosity measured in cP.

### BACKGROUND OF THE INVENTION

In modern mashing processes, enzymes are often added as a supplement when mashing malt and adjunct grist. Enzymes may also be applied in mashing of well modified malts with high enzyme content to increase the extract recovery and the amount of fermentable sugars, as well as accelerate the overall conversion time.

Matthews et al., 2001, Journal of Institute of brewing 107 (3) pp 185-194, discloses preparation of a low carbohydrate beer by mashing a malt grist with a glucoamylase, which is derived from Aspergillus niger.

WO 2012/140075 describes the use of a glucoamylase from Penicillium oxalicum when mashing a malt and adjunct grist.

However, there still exists a need for methods that will optimize the use of the raw material in the production of brewer's wort in adjunct brewing, e.g. brewing using Sorghum.

### SUMMARY OF THE INVENTION

The present inventors have surprisingly found that alpha-amylases with calculated pI in the range from 4.0 to 4.6 are particularly efficient in liquifying Sorghum, even in Sorghum with high content of condensed tannins (Sorghum comprising at least 10 mg catechin equivalents/g Sorghum).

Thus, the invention relates in a first aspect to a method of producing a brewer's wort comprising the steps
a) Providing a mash from a grist comprising unmalted Sorghum;
b) Adding an alpha-amylase to the mash;
c) Heating the mash to the pasting temperature of the unmalted Sorghum starch, and
d) Producing a wort,
wherein the alpha amylase has a calculated pI in the range of from 4.0 to 4.6.

The invention relates in a second aspect to the use of an alpha-amylase with a calculated pI in the range of from 4.0 to 4.6, for brewing, in particular Sorghum brewing.

In a third aspect, the invention relates to a beer produced according to the method.

### DETAILED DESCRIPTION OF THE INVENTION

### Definitions

The term “grist” is understood as the starch- or sugar-containing material that is the basis for beer production.

The term “malt” is understood as any malted cereal grain.

The term “adjunct” is understood as the part of the grist which is not malt.

The term “mash” is understood as a starch containing slurry of the grist comprising crushed grain, optionally other starch containing material, or a combination thereof, steeped in water to make wort.

The term “mashing” is the process of converting starch in the mash into fermentable and un-fermentable sugars.

The term “wort” is understood as the unfermented liquor run-off, following extracting the grist during mashing.

The term “identity” is the relatedness between two amino acid sequences. For purposes of the present invention, the degree of identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends in Genetics 16:276-277), preferably version 5.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment).

### Method of Brewing

The invention relates in a first aspect to a method of producing a brewer's wort comprising the steps:
a) Providing a mash from a grist comprising unmalted Sorghum;
b) Adding an alpha-amylase to the mash;
c) Heating the mash to the pasting temperature of the unmalted Sorghum starch; and
d) Producing a wort
wherein the alpha-amylase has a calculated pI in the range from 4.0 to 4.6.

The inventors have surprisingly shown that the above method leads to particularly good liquefication of unmalted Sorghum, even where the Sorghum has high content of condensed tannins. Thus, the method of the invention improves yield where unmalted Sorghum is used in brewing as it leads to better usage of the raw material. The method of the invention further makes it possible to use Sorghum cultivars not previously possible to be used in brewing (Sorghum containing high content of condensed tannins, e.g. “bird-proof” cultivars of Sorghum). Even further, the method of the invention provides an advantage as brewers may use the same method regardless of tannin content in Sorghum, obviating the need for switching or running alternative methods.

### Method of Producing a Brewer's Wort and a Beer According to the Invention

Conventional machinery, equipment, and materials can be used during mashing.

The grist is mixed with water prior to mashing. The water may, before being added to the grist, be preheated in order for the mash to attain the desired mash temperature at the moment of mash forming.

If the temperature of the formed mash is below the desired mashing temperature, additional heat is preferably supplied in order to attain the desired start process temperature.

Preferably, the desired start mashing temperature is attained within 15 minutes, or more preferably within 10 minutes, such as within 9, 8, 7, 6, 5, 4, 3, 2 minutes or even more preferably within 1 minute after the mash forming, or most preferably the desired mashing temperature is attained at the mash forming.

The alpha amylase may be added to the mash ingredients, e.g., the water and/or the grist before, during or after forming the mash, or at any time during the mashing.

The alpha amylase may be added as an enzyme composition. Such enzyme compositions may comprise one or more enzymes. The enzyme composition, in addition to the enzyme(s), may also further comprise at least one other substance, for example but not limited to buffer, surfactants etc. The enzyme compositions may be in any form, for example, solid, liquid, emulsion, gel, or paste. Such forms are known to the person skilled in the art.

During the mashing process, starch extracted from the grist is gradually hydrolysed into fermentable sugars, smaller dextrins, and glucose.

The mashing comprises as a step of heating, suitable to achieve pasting and/or gelatinization.

The method of the invention comprises a step of heating the mash to the pasting temperature of the unmalted Sorghum starch. This is done in order to achieve at least pasting and/or gelatinization of the Sorghum starch. RVA determined pasting temperature can be used as an indication of starch gelatinization. The pasting/gelatinization temperature of Sorghum is known in the art, and is typically in the range from 65 to 78 °C.

Thus, this heating step may be at a temperature of at least 60 °C, at least 65 °C, or at least 70 °C, at least 75 °C, at least 78 °C, at least 79 °C or at least 80 °C; or for example at a temperature in the range of from 60 to 95 °C, such as from 62 to 95 °C, 67 to 95 °C, 70 to 95 °C, 72 to 95 °C, 75 to 95 °C, 77 to 95 °C, 80 to 95 °C, 82 to 95 °C or 85 to 95 °C, for a time which is suitable to achieve pasting.

The heating step may for example be at any temperature interval mentioned above and for a time of at least 5 minutes, such as at least 7 min, at least 9 min, at least 10 min, at least 12 min, at least 15 min, at least 20 min; or for example for a time in the range of from 2 to 90 min, such as 2 to 85 min, 2 to 80 min, 2 to 75 min, 2 to 70 min, 2 to 65 min, 2 to 60 min.

Particular embodiments relate to an incubation at at least 60 °C for at least 5 mins, or for example at at least 65 °C for at least 10 mins, or at a temp in the range of from 60 to 95 °C for a time in the range of from 5 to 90 mins; or a temperature in the range of from 65 to 90 °C for a time in the range of from 5 to 80 mins.

Further particular embodiments relate to where the heating step is at a temperature in the range of from 70 to 95 °C for a time in the range of from 5 to 40 mins.

After mashing, when all the starch has been broken down, it is necessary to separate the liquid extract (the wort) from the solids (spent grains).

Wort separation, lautering, is important because the solids contain large amounts of protein, poorly modified starch, fatty material, silicates, and polyphenols (tannins).

The extract retained in the spent grain after collection of the first wort may also be washed out by adding hot water on top of the lauter cake. This process is called sparging. The hot water flows through the spent grain and dissolves the remaining extract. The diluted wort is called second wort.

Following the separation of the wort from the spent grains, typically the wort is boiled, optionally after addition of hops.

After cooling and removal of the precipitates, the wort is aerated, and yeast is added.

The yeast applied may be any yeast suitable for beer brewing, especially yeasts selected from Saccharomyces such as S. cerevisiae and S. uvarum, including natural or artificially produced variants of these organisms. A person skilled in the art will be able to select a suitable yeast.

After a main fermentation, lasting typically 5-10 days, most of the yeast is removed, and the so-called green beer is stored at a low temperature, typically at 0 °C-5 °C for 1 to 12 weeks. After maturation step, beer is typically filtered and boiled. Optionally, the beer may be carbonated.

Further information on conventional brewing processes may be found in “Technology Brewing and Malting” by Wolfgang Kunze of the Research and Teaching Institute of Brewing, Berlin (VLB), 2nd revised Edition 1999, ISBN 3-921690-39-0.

Any beer type may be produced from the wort according to the present invention. Preferred beer types comprise ales, strong ales, stouts, porters, lagers, bitters, export beers, malt liquors, happoushu, high-alcohol beer, low-alcohol beer, low-calorie beer, or light beer.

The wort may also be processed to be used as syrups. It may also be used to produce non-alcoholic beverages. These processes are well known to a person skilled in the art.

### Sorghum

The method according to the present invention comprises providing a mash from a grist comprising unmalted Sorghum.

Sorghum belongs to the family of grasses (Poaceae) and comes in different cultivars and varieties that are adapted for various areas and uses. Insight into the specific physicochemical properties and how physical- and biochemical treatments affect them is the basis to assure an efficient processability in a brewery and hence good beer quality.

As mentioned, the method of brewing according to the invention provides a robust method for brewing Sorghum, regardless of condensed tannin content. Thus, the unmalted Sorghum may be one or more species selected from the following:
1. Sorghum amplum
2. Sorghum angustum
3. Sorghum arundinaceum
4. Sorghum bicolor
5. Sorghum brachypodum
6. Sorghum bulbosum
7. Sorghum burmahicum
8. Sorghum controversum
9. Sorghum drummondii
10. Sorghum ecarinatum
11. Sorghum exstans
12. Sorghum grande
13. Sorghum halepense
14. Sorghum interjectum
15. Sorghum intrans
16. Sorghum laxiflorum
17. Sorghum leiocladum
18. Sorghum macrospermum
19. Sorghum matarankense
20. Sorghum nitidum
21. Sorghum plumosum
22. Sorghum propinquum
23. Sorghum purpureosericeum
24. Sorghum stipoideum
25. Sorghum timorense
26. Sorghum trichocladum
27. Sorghum versicolor
28. Sorghum virgatum

In particular embodiments, the Sorghum is Sorghum bicolour, in further particular embodiments, the Sorghum is Sorghum bicolor (L.) Moench.

The invention relates in some embodiments to where the grist comprises at least 1% unmalted Sorghum, such as at least 2% unmalted Sorghum, such as at least 4% (w/w) unmalted Sorghum, such as at least 5% (w/w) unmalted Sorghum; or for example the grist comprises in the range of from 1 to 100% unmalted Sorghum, such as from 1 to 90%, 2 to 90%, 2 to 85%, 2 to 80%, 2 to 85% % unmalted Sorghum (w/w).

The method according to the invention provides improved liquefaction of Sorghum.

Thus, some embodiments relate to the method according to the invention wherein the grist comprises at least 10% w/w unmalted Sorghum, such as at least 15% w/w unmalted Sorghum, at least 20% w/w unmalted Sorghum, as at least 25% w/w unmalted Sorghum, at least 30% w/w unmalted Sorghum, at least 35% w/w unmalted Sorghum; such as the grist comprises at least 40% (w/w) unmalted Sorghum, e.g. the grist comprises at least 45% (w/w) unmalted Sorghum; e.g., the grist comprises at least 50% (w/w) unmalted Sorghum; e.g. the grist comprises at least 55% (w/w) unmalted Sorghum; e.g., the grist comprises at least 60% (w/w) unmalted Sorghum; e.g., the grist comprises at least 70% (w/w) unmalted Sorghum; e.g. the grist comprises at least 75% (w/w) unmalted Sorghum; e.g. the grist comprises at least 80% (w/w) unmalted Sorghum; e.g., the grist comprises at least 85% (w/w) unmalted Sorghum; e.g., the grist comprises at least 90% (w/w) unmalted Sorghum; e.g., the grist comprises at least 95% (w/w) unmalted Sorghum.

In one embodiment, the grist consists of unmalted Sorghum.

In some embodiments, the grist comprises both unmalted Sorghum and malted Sorghum.

In some embodiments, the grist comprising unmalted Sorghum may further comprise one or more malted or unmalted cereal and/or pseudocereal grains, such as barley, wheat, rye, buckwheat, amaranth, quinoa, millet, oat, Sorghum or combinations thereof.

In one embodiment, the grist comprises or consists of unmalted Sorghum and malted barley.

In some embodiments, the grist may comprise further adjunct, such as raw and/or refined starch and/or sugar containing material derived from one or more of wheat, rye, oat, maize, rice, milo, millet, Sorghum, potato, sweet potato, cassava, tapioca, sago, banana, sugar beet and/or sugar cane.

In a preferred embodiment, the grist comprises or consists of unmalted Sorghum and cassava.

Preferably, said further adjunct(s) have high pasting or gelatinization temperature, such as a temperature of at least 60 °C. More particularly, these adjuncts have a high onset pasting or gelatinization temperature.

Adjunct may also comprise readily fermentable carbohydrates such as sugars or syrups and they may be added to the mash before, during or after the mashing process of the invention, but is preferably added after the mashing process.

Prior to forming the mash, the adjunct is preferably milled and most preferably dry or wet milled.

Sorghum varieties vary in the amount of condensed tannins they contain. Condensed tannins (proanthocyanidins, polyflavonoid tannins, catechol-type tannins, pyrocatecollic type tannins, non-hydrolyzable tannins or flavolans) are polymers formed by the condensation of flavans.

For example, the total amount unmalted Sorghum in the grist may have a content of condensed tannins, as determined by vanillin method (see materials and methods) in the range of from 0.2 mg to 90 mg catechin equivalents per g Sorghum, for example in the range of from 1 to 90, from 3 to 90, from 5 to 90, from 6 to 90, from 10 to 90, from 15 to 90, from 18 to 90, from 20 to 90, from 25 to 90, from 30 to 90, from 35 to 90, from 40 to 90, from 45 to 90, from 50 to 90, from 50 to 90, from 60 to 90, from 65 to 90, from 70 to 90 mg catechin equivalents per g Sorghum, as determined by vanillin assay (see Materials and Methods).

Sorghum are sometimes divided into three groups based on tannin content (Hahn and Rooney, Cereal Chem., 63 (1).4-8) 1985). Group I Sorghums do not contain tannins; while Group II and Group III do. Group III contains more tannins than do Group II Sorghums. Group III Sorghum is sometimes known as “birdproof cultivars”.

So-called birdproof cultivars and varieties of Sorghum show superior agronomic characteristics and are therefore the preferred choice of farmers in regions, where the grain is exposed to migration birds. The high content of condensed tannins in these varieties renders the Sorghum less attractive to birds. However, this high content in condensed tannins in birdproof cultivar inhibits amylolytic hydrolysis in the mashing step of brewing, and they display significant processing difficulties and are typically only used at limited inclusion rates in the grist composition (such as below 20% w/w of grist, or below 40% w/w of grist).

The method according to the invention is useful for liquefaction of all Sorghum, and in particular, surprisingly, is efficient on Sorghum having high content of condensed tannins.

Thus, some embodiments relate to the method according to the invention, wherein the unmalted Sorghum comprises or consists of Sorghum having high content of condensed tannins.

As used herein, the term “high content of condensed tannins” refers to Sorghum having at least 10 mg catechin equivalents/g Sorghum.

In one embodiment, the unmalted Sorghum may comprise or consist of one or more Group III Sorghum.

In one embodiment, the unmalted Sorghum may comprise or consist of one or more Group II Sorghum.

In one embodiment, the unmalted Sorghum does not comprise Sorghum from Group I.

In one embodiment the unmalted Sorghum provided consists of Sorghum from Group II and/or Group III.

In one embodiment the unmalted Sorghum provided consists of Sorghum having at least 10 mg catechin equivalents/g Sorghum.

### Enzymes

### Alpha-Amylase (EC 3.2.1.1)

The inventors have surprisingly shown that alpha-amylases with a calculated pI in the range from 4.0 to 4.6 are particularly efficient at liquefying Sorghum, in particular Sorghum having high content of condensed tannins.

Thus, the invention relates to a method of producing a brewer's wort comprising the steps
a) Providing a mash from a grist comprising unmalted Sorghum;
b) Adding an alpha-amylase to the mash;
c) Heating the mash to the pasting temperature of the unmalted Sorghum, and
d) Producing a wort,
wherein the alpha amylase has a calculated pI in the range of from 4.0 to 4.6.

Some embodiments relate to the method according to the invention wherein the alpha-amylase according the embodiment has a calculated pI in the range of from 4.0 to 4.6, such as 4.1 to 4.6, or for example from 4.2 to 4.5.

In some embodiments of the invention, the alpha amylase has at least 60% identity, such as at least 65% identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% identity to the sequence shown in SEQ ID NO: 1.

In some embodiments of the invention, the alpha amylase has at least 60% identity, such as at least 65% identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% identity to the sequence shown in SEQ ID NO: 5

In some embodiments of the invention, the alpha amylase has at least 60% identity, such as at least 65% identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% identity to the sequence shown in SEQ ID NO:9.

The alpha amylase according to the invention is suitable for use at or above pasting temperature for Sorghum.

In some embodiments, the alpha amylase according to the invention displays at least 50% residual activity at pasting temperature after 10 minutes.

In some embodiments, the alpha amylase according to the invention such as displays at least 50% residual activity after 10 minutes at a temperature of 60 °C, 65 °C, 70 °C, 75 °C, 78 °C, 79 °C, 80 °C, 85 °C, 90 °C or 95 °C; or for example at a temperature in the range of from 60 to 95 °C, such as from 62 to 95 °C, 67 to 95 °C, 70 to 95 °C, 72 to 95 °C, 75 to 95 °C, 77 to 95 °C, 80 to 95 °C, 82 to 95 °C or 85 to 95 °C.

In particular embodiments, the alpha amylase according to the invention displays at least 50% residual activity after 10 minutes at a temperature of at least 78 °C, such as at a temperature of at least 80 °C, at least 85 °C or at least 90 °C.

The brewer may choose to employ one or more further alpha-amylases. Thus, some embodiments relate to the method according to the invention, wherein one or more further alpha-amylases are added.

The one or more further alpha-amylases are not limited, and the selection of such amylases is within the skill of the art.

Such one or more further alpha amylases may be added prior to, at the same time as, or after the addition of the alpha-amylase according to the invention.

Some embodiments relate to where the one or more further alpha amylases are selected from the group consisting of Amylex 6T (Dupont), Termamyl SC (Novozymes), Fortiva Revo (Novozymes).

Particular embodiments relate to the method according to the invention wherein alpha amylase comprises ENZ 1 and said further alpha-amylases is selected from one or more of ENZ 2, ENZ 3, ENZ 4, ENZ 6, ENZ 7, ENZ 8, ENZ 10, ENZ 11, ENZ 12, molecules having at least 70% sequence identity to these, and combinations thereof.

Other particular embodiments relate to the method according to the invention wherein adding alpha amylase comprises adding ENZ 1, ENZ 5 and/or ENZ 9, or a molecule having at least 60% sequence identity to them, or combinations thereof; and further comprises adding one or more further alpha-amylases, such as one or more of commercially available alpha-amylases, such as Amylex 6T (Dupont), Fortiva Revo (Novozymes), Termamyl (Novozymes).

Other particular embodiments relate to the method according to the invention wherein adding alpha amylase comprises adding ENZ 1 or a molecule having at least 60% sequence identity to SEQ ID NO: 1, and further comprises adding one or more further alpha-amylases, such as one or more of commercially available alpha-amylases, such as Amylex 6T (Dupont), Fortiva Revo (Novozymes), Termamyl SC (Novozymes).

In some embodiments, alpha amylase is added in a concentration of 0.5 to 500 mg of enzyme protein (EP) per kg of total weight of the grist, e.g., 5 to 500 mg of enzyme protein (EP) per kg of total weight of the grist, e.g., 10 to 100 mg of enzyme protein (EP) per kg of total weight of the grist.

### Glucoamylase (EC 3.2.1.3)

Some embodiments relate to the method according to the invention wherein one or more further enzymes are added.

Glucoamylases (Glucan 1,4-alpha-glucosidase) are enzymes which catalyze the hydrolysis of terminal (1-4)-linked alpha-D-glucose residues successively from non-reducing ends of the chains with release of beta-D-glucose.

In one embodiment, the method of the invention further comprises addition of one or more glucoamylases.

For example, said one or more glucoamylases may be for example as described in WO2019219601, hereby incorporated by reference.

In another preferred embodiment, one or more further enzyme(s) is added to the mash, said enzyme(s) including but not limited to isoamylase, protease, cellulase, glucanase, laccase, xylanase, lipase, phospholipolase, phytase, phytin, and esterase.

In one embodiment of the method, the further enzyme added includes but is not limited to a protease.

In one embodiment of the method, the further enzyme added includes but is not limited to a cellulase.

In one embodiment of the method, the further enzyme added includes but is not limited to a xylanase.

In one embodiment of the method, the further enzyme added includes but is not limited to a lipase.

In a second aspect, the invention relates to the use of an alpha-amylase with a calculated pI in the range from 4.0 to 4.6, for use in brewing, in particular in brewing of beer comprising Sorghum.

Parameters described in relation to the method of the invention above also relate to the aspect of use.

Specific embodiments of this aspect relate to an alpha amylase for use in brewing of beer comprising Sorghum, wherein said alpha amylase is selected from the group consisting of ENZ1, ENZ 5 and ENZ 9 and sequences with homology of at least 60% thereto.

The method of the invention liquefies the Sorghum mash and displays little to no set-back. Indeed in experimental conditions, negative set-back is shown. This facilitates the handling of the mash.

Thus, a further aspect of the invention relates to a mash produced by the method according to the invention as described herein, comprising an alpha amylase according to the invention.

The invention in a further aspect relates to a beer produced according to the method of the invention.

### EMBODIMENTS

The following is a non-exhaustive list of embodiments of the invention:
1. A method of producing a brewer's wort comprising the steps
   a) Providing a mash from a grist comprising unmalted Sorghum;
   b) Adding an alpha-amylase to the mash;
   c) Heating the mash to the pasting temperature of the unmalted Sorghum starch, and
   d) Producing a wort,
   wherein the alpha amylase has a calculated pI in the range of from 4.0 to 4.6.
2. The method according to embodiment 1 wherein the grist comprises at least 20% (w/w) unmalted Sorghum.
3. The method according to any of the preceding embodiments wherein the grist consists of unmalted Sorghum.
4. The method according to any of the preceding embodiments, wherein the unmalted Sorghum comprises Sorghum with content of condensed tannins in the range of from 0.2 to 90 mg catechin equiv./g Sorghum.
5. The method according to any of the preceding embodiments, wherein the alpha amylase is selected from the group comprising SEQ ID NO:1, SEQ ID NO:5 and SEQ ID NO: 9 and molecules having 60% identity to them.
6. The method according to any of the preceding embodiments, wherein the alpha-amylase has at least at least 60% identity to the sequence shown in SEQ ID NO: 1
7. The method according to any of the preceding embodiments wherein the alpha-amylase has at least at least 60% identity to the sequence shown in SEQ ID NO: 5.
8. The method according to any of the preceding embodiments 1-3, wherein the alpha-amylase has at least at least 60% identity to the sequence shown in SEQ ID NO:9.
9. The method according to any of the preceding embodiments, further comprising adding a further alpha amylase.
10. The method according to any of the preceding embodiments, further comprising adding a glucoamylase.
11. The method according to any of the preceding embodiments, further comprising adding a protease.
12. The method according to any of the preceding embodiments, further comprising adding a xylanase.
13. The method according to any of the preceding embodiments, further comprising adding a lipase.
14. The method according to any of the preceding embodiments, further comprising adding a cellulase.
15. The method according to any of the preceding embodiments, wherein the alpha-amylase is added in an amount of 0.5 to 500 mg enzyme protein per kg grist.
16. The method according to any of the preceding embodiments, wherein the glucoamylase is added in an amount of 1 to 1000 mg enzyme protein per kg grist.
17. The method according to any of the preceding embodiments, wherein the total mashing time is less than 130 minutes.
18. The method according to any of the preceding embodiments, wherein the wort is converted to beer.
19. The use of an alpha-amylase with a calculated pI in the range from 4.0 to 4.6, for use in brewing.
20. The use according to embodiment 19 wherein said alpha amylase displays at least 50% residual activity after 10 minutes at a temperature of at least 78 °C.
21. The liquefied mash produced by the method according to any of the preceding embodiments, comprising an alpha amylase with a calculated pI in the range from 4.0 to 4.6.
22. A beer produced by the method according to any of the preceding embodiments 1-20.

### EXAMPLES

### Materials and Methods

### Sequences

Molecules used in the examples are described in Table 1 below.

TABLE 1
Molecules described in this application and sequence ID nos.

| SEQ ID NO | ENZ | PI | CLASS |
|---|---|---|---|
| SEQ ID NO 1 | ENZ 1 | 4.2 | GH13_1 + CBM20 |
| SEQ ID NO 2 | ENZ 2 | 5.6 | GH13_5 |
| SEQ ID NO 3 | ENZ 3 | 5.6 | GH13_5 |
| SEQ ID NO 4 | ENZ 4 | 5.5 | Gh13_5 |
| SEQ ID NO 5 | ENZ 5 | 4.3 | GH13_1 |
| SEQ ID NO 6 | ENZ 6 | 3.9 | GH13_1 + CBM20 |
| SEQ ID NO 7 | ENZ 7 | 5.2 | GH13_M43 |
| SEQ ID NO 8 | ENZ 8 | 4.7 | GH13_28 + CMB20 |
| SEQ ID NO 9 | ENZ 9 | 4.5 | GH13_28 |
| SEQ ID NO 10 | ENZ 10 | 6.95 | GH13_1 |
| SEQ ID NO 11 | ENZ 11 | 5.4 | GH13 |
| SEQ ID NO 12 | ENZ 12 | 4.8 | GH13_1 |
| | ENZ 13 | 5.5 | GH_13 variant |
| | ENZ 14 | 6.5 | GH13_7 |

### Sorghum

Sorghum (Sorghum bicolor (L) Moench) contains a wide variety of phenolic compounds and their levels and types depend on the genotype. Sorghums with a pigmented testa contain condensed tannins, which are the main phenolic compounds in those genotypes and are concentrated in the pigmented testa.

Sorghum varieties are divided into three groups. Type I Sorghums do not have a pigmented testa and contain low levels of phenolic compounds and no tannins. Type II and III Sorghums both have a pigmented testa and contain tannins, but the tannins in type II Sorghums are extracted with acidified methanol (1% HCL) while those in type III Sorghums are extracted with either methanol or acidified methanol. Typically, Type II Sorghums contain lower tannin levels than Type III Sorghums.

However, the colour of the Sorghum seed does not appear to directly correlate with the colour of the testa and thereby the amount of condensed tannin.

The vanillin-HCL assay involves the condensation of vanillin with monomeric flavanols and their oligomers to form a red adduct that absorbs at 500 nm. The amount of condensed tannins in different Sorghums was tested by the colorimetric vanillin HCL assay described in Dykes L. Tannin Analysis in Sorghum Grains. Methods Mol Biol. 2019; 1931:109-120. doi: 10.1007/978-1-4939-9039-9_8. PMID: 30652286.

TABLE 2
Tannin content of Sorghum

| | | Type II and III | | | |
|---|---|---|---|---|---|
| | | Mg catechin | | | |
| | | equiv./g | | Type III | |
| | | sorghum | | Without | |
| Number | Sorghum | with HCL | STD | HCL | STD |
| SS-2020- | Red sorghum | 74.20 | 0.85 | 63.60 | 2.41 |
| 00023 | Kenya, | | | | |
| SS-2020- | White sorghum | 12.23 | 0.30 | 0.70 | 0.07 |
| 00021 | Kenya, | | | | |
| SS-2020- | Red sorghum | 46.38 | 0.97 | 36.13 | 2.51 |
| 0006 | Burundi, | | | | |
| SS-2020- | Red sorghum, | 0.38 | 0.92 | 1.38 | 0.36 |
| 00012 | Nigeria | | | | |
| SS-2018- | Red sorghum | 28.48 | 1.94 | 26.34 | 2.68 |
| 00055 | Kenya, | | | | |

### Liquefaction Assay

Screening of alpha amylases suitable to liquify Sorghum high content of condensed tannins was done by testing enzymes in simulated liquefaction process. The trials used in situ monitoring of suspension viscosity by a Rapid Visco Analyzer RVA 4500 (PerkinElmer, Stockholm, Sweden; former Perten Instruments).

Sorghum was milled using a Lab mill 3100 and retention sieve 0.8 mm (mm nominal diameter).

21 g water was mixed with 7 g grist (water to grist ratio of 3 to 1). Calcium ions were added to the mash water in an amount of 85 ppm.

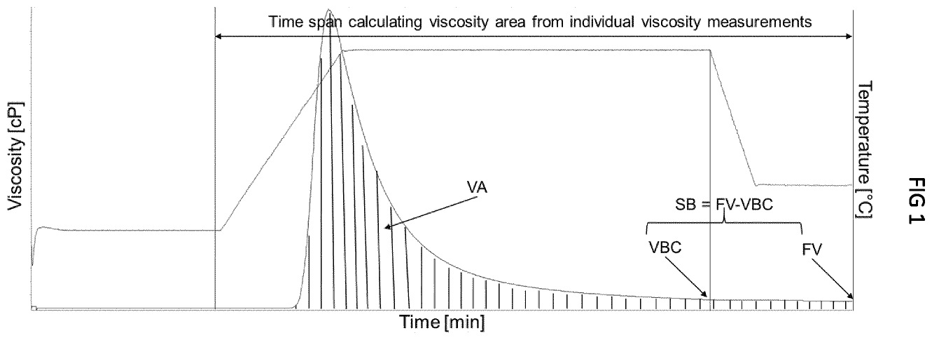

The liquefaction processes are detailed below, q.v. See also FIG. 1 for visual overview of process.

Enzyme performance was evaluated based on the final viscosities obtained at the end of the process, as well as the area of the integration of the individual viscosity measurements over the process time, from 10 mins after start to the end (See FIG. 1, and Brief description of figures, FIG. 1).

The set-back was also considered. Set-back was measured as the final viscosity, minus the viscosity at the end of the holding phase after cooling to 65 °C (see FIG. 1). The cooling simulates a typical subsequent temperature in a process in which saccharification of the liquified starch is desirable, e.g. production of wort for beer production). This set-back measurement gives an indication to what degree the hydrolysed starch can reorganize its structure (=retrogradation).

### X-Simulated Liquefaction Process(es) on RVA

Temperature profiles (including applied stirring conditions); viscosity measurement were conducted every second throughout the whole liquefaction process. See Table 3A below for the conditions used when holding temperature as 90 °C. See also FIG. 1 for overview of liquefaction process.

TABLE 3A
Overview of liquefaction process conditions

| Time (hh:mm:ss) | Function Type | Value |
|---|---|---|
| 00:00:00 | Temp | 60 |
| 00:00:00 | Speed | 960 |
| 00:00:15 | Speed | 100 |
| 00:10:15 | Temp | 60 |
| 00:20:17* | Temp | 90 |
| 00:36:57 | Temp | 90 |
| 00:39:27 | Temp | 65 |
| 00:44:27 | Temp | 65 |
| 00:44:45 | End | 65 |

*see Table 3B for start times of Holding temp for the different holding temperatures.

TABLE 3B
Overview of liquefaction process conditions
for various holding temperatures

| Holding temp | |
|---|---|
| (degree C) | Time |
| 90 | 00:20:17 |
| 87.5 | 00:19:27 |
| 85 | 00:18:37 |
| 82.5 | 00:17:47 |
| 80 | 00:16:57 |

### Calculation of pI

Briefly, the calculated isoelectric point for a protein is a theoretical value calculated from its amino acid composition, assuming that no electrostatic interactions change the propensity for ionization. The calculated pI used herein was calculated by running pepstats command from EMBOSS 6.6.0 with the Epk.dat file from 6.3.1. This pI calculation does not take predicted signal peptide, or propeptide removal into account.

EMBOSS 6.6.0 is available from http://emboss.sourceforge.net/for download and installation on a LINUX computer. The Epk.dat file from 6.3.1 is named: “pepstats-pkfile Epk_emboss_6_3_1.dat-filter-sequence SEQUENCEFILE”, and contains:
#pk values for amino acids
#O=Ornithine J=Hydroxyproline or Leucine/Isoleucine
#
#Amino acid pK
Amino 8.6
Carboxyl 3.6
#Acidic
#B converted to 5.5/9.8 D, 4.3/9.8 N according to Dayhoff frequencies
#Z converted to 6.0/9.9 E, 3.9/9.8 Q according to Dayhoff frequencies
C 8.5
D 3.9
E 4.1
#Basic
H 6.5
K 10.8
R 12.5
Y 10.1
===

### Example 1. Evaluation of Alpha-Amylases on Liquefaction of Sorghum

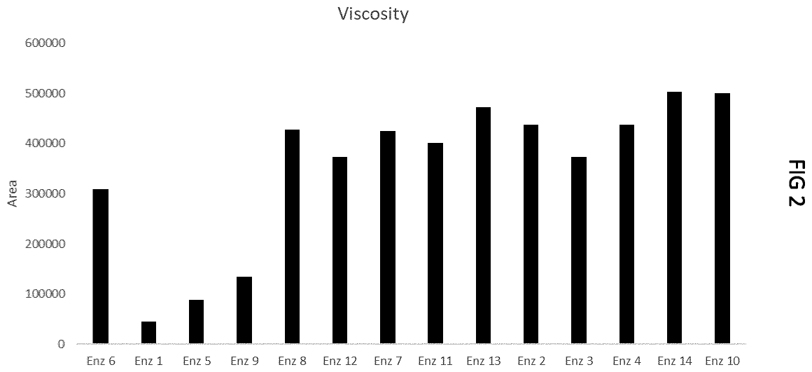

A number of enzymes were screened for their performance in liquefying Sorghum using the liquefaction process described above. The results are shown in the Table 4 and also in FIG. 2.

Mixture of Sorghum SS-2020-00023 (74.2 mg catechin equivalent/g Sorghum, example of a Sorghum with high content condensed tannins), water and enzyme were prepared according to materials and methods. Enzymes were dosed at 13.3 mg enzyme protein/kg Sorghum grist of the respective alpha-amylases. Liquefaction was performed using liquefaction profile with holding temperature of 80 °C (see Tables 3A and 3B).

The liquefaction process was monitored by in-situ viscosity measurement of the Sorghum grist/water suspension using a Perten Rapid Visco Analyzer (RVA). Performance was evaluated by integrating the area underneath the viscosity curve obtained from the liquefaction process, as well as the final viscosity at the end of liquefaction process (after cooling to 65 °C, simulating a typical subsequent temperature in a process in which saccharification of the liquified starch is desirable, e.g. production of wort for beer production) and the set-back (final viscosity at end of process minus viscosity before cooling from holding temperature to final temperature).

Results are shown in Table 4 and FIGS. 2, 3 and 4.

TABLE 4
Different alpha-amylases liquify sorghum to varying degrees

| | | Area | Final | |
|---|---|---|---|---|
| | | under the | viscosity | |
| Number | SEQ ID | curve | [cP] | Set back |
| ENZ 1 | SEQ ID NO 1 | 44636 | 63 | −28 |
| ENZ 2 | SEQ ID NO 2 | 437258 | 19922 | 10128 |
| ENZ 3 | SEQ ID NO 3 | 372621 | 15477 | 8282 |
| ENZ 4 | SEQ ID NO 4 | 437258 | 6609 | 2987 |
| (Amylex 6T) | | | | |
| ENZ 5 | SEQ ID NO 5 | 88092 | 2172 | 622 |
| ENZ 6 | SEQ ID NO 6 | 309416 | 14990 | 7483 |
| ENZ 7 | SEQ ID NO 7 | 424271 | 19985 | 8721 |
| ENZ 8 | SEQ ID NO 8 | 427305 | 23221 | 13463 |
| ENZ 9 | SEQ ID NO 9 | 134904 | 3618 | 2048 |
| ENZ 10 | SEQ ID NO 10 | 499479 | 28669 | 16995 |
| ENZ 11 | SEQ ID NO 11 | 401550 | 23035 | 14242 |
| ENZ 12 | SEQ ID NO 12 | 372699 | 15124 | 5242 |
| ENZ 13 | Experimental | 471713 | 26395 | 14896 |
| ENZ 14 | Experimental | 503419 | 28304 | 15790 |

### Area Underneath Viscosity Curve

Out of the tested 14 alpha-amylase enzymes, one sub-group showed superior performance and were able to extensively liquefy Sorghum containing high content of condensed tannins. Enzymes ENZ 1, ENZ 5 and ENZ 9 showed significant reduction of the integrated area underneath the viscosity curve and final viscosity when compared with tested samples. Their performance was distinctly different from the other tested alpha amylases (see FIG. 2).

Surprisingly, it was noted that all enzymes which showed very good or good performance in liquifying Sorghum with high content of condensed tannins, had a calculated isoelectric point (pI) between 4.0 and 4.6.

The effect cannot be attributed to the presence of CBM20 domain in ENZ 1. Both ENZ 6 and ENZ 8 comprise CBM20 domains, yet neither display the improved liquefaction performance of ENZ 1, ENZ 5 and ENZ 9.

ENZ 1 was particularly effective. Treatment with ENZ1 resulted in a fully liquid mash (Sorghum mash after liquefaction being liquid) at a dosage of 13.3 mg enzyme protein/kg Sorghum when holding temperature during the liquefaction process was 80°, see Table 4, as well as FIG. 2.

### Final Viscosity

The findings from Area under the curve supported that the enzymes ENZ 1, ENZ 5 and ENZ 9 show best performance.

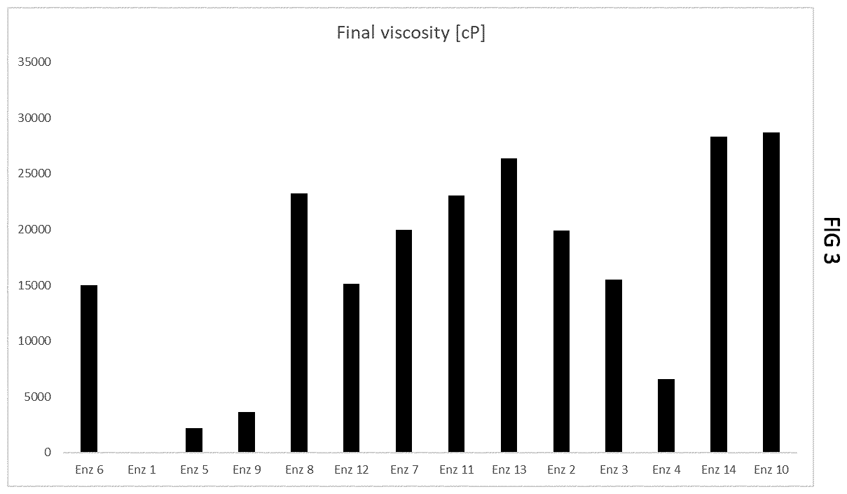

Outside of the three best performing enzymes, ENZ 4 (Amylex 6T TM, Dupont) displayed lowest final viscosity; However, the area under the viscosity curve for that enzyme (see Table 4 and FIGS. 2 and 3) indicates that it takes much longer time for this enzyme to achieve this viscosity. In contrast, the enzymes ENZ 1, ENZ 5 and ENZ 9 achieve the lowest final viscosity, together with the lowest area under the curve of viscosity. This underscores the superior performance of these enzymes.

### Example 2. Evaluation of Alpha-Amylases Liquefaction on Sorghum-Set Back

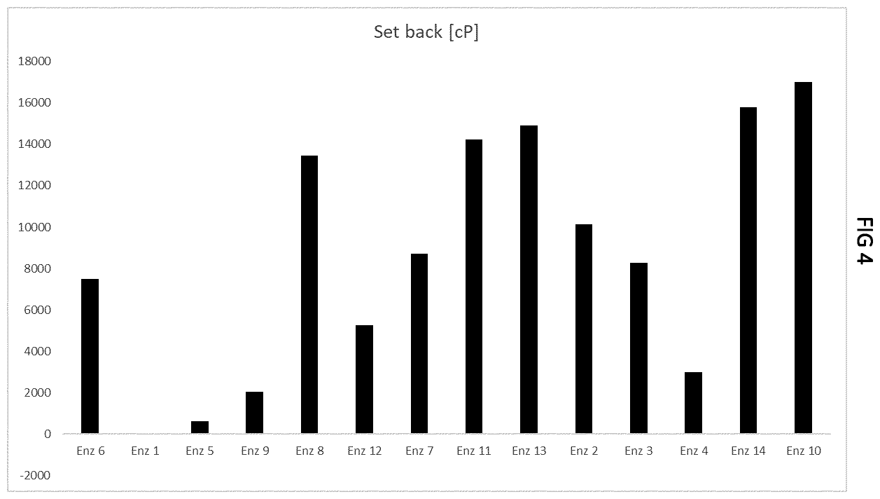

Increase in viscosity for the samples in Example 1 (set-back) after cooling from holding (80 °C) to final (65 °C) temperature and 5 minutes rest as it is shown in Table 4, set-back and FIG. 4.

Setback, also known as starch retrogradation, is the phenomenon when starch which is not fully degraded in the liquefaction process, re-assembles to some degree after liquefaction process—this reassembly is reflected in increased viscosity on cool-down. Higher set-back means increased degree of starch retrogradation.

The three best performing enzymes (ENZ 1, ENZ 5 and ENZ 9) in Example 2 also showed good performance here, i.e. they display low to no setback.

ENZ 1 displayed a negative set-back value.

### Example 3. Evaluation of ENZ 1 on Various Sorghum Types

The performance of ENZ 1 on different Sorghums, which contain different concentrations of condensed tannins, was evaluated.

Sorghum from different sources were subjected to liquefaction process as described in Materials and methods, in presence of 9 mg acid alpha-amylase ENZ 1/kg Sorghum grist.

As shown in Table 5, Sorghum varieties with lower concentrations (0.38, 12.23, 28.48 and 46.38 mg catechin equivalent/g Sorghum) were tested in a liquefaction process with 80 °C holding temperature. ENZ 1 showed similar liquefaction performance for all Sorghums, and all tested Sorghums could be completely liquified when the holding temperature was set to 80 °C. See Table 2 for Sorghums.

TABLE 5
Area under the curve, final viscosity and Setback

| | Mg | Area underneath | | |
|---|---|---|---|---|
| Sorghum | Catechin | Viscosity curve from | Final | Set- |
| sample | equiv./g | 10 mins to end | viscosity | back |
| SS-2018-0055 | 28.47 | 45156 | 123 | −49 |
| SS-2020-0006 | 46.38 | 43748 | 51 | 23 |
| SS-2020-0012 | 0.38 | 47937 | 151 | −72 |
| SS-2020-0021 | 12.23 | 60503 | 234 | −130 |
| SS-2020-0023 | 74.20 | 58816 | 103 | −44 |

The area underneath the viscosity curves as well as the final viscosities showed variation for the different Sorghum mashes (see Table 5), however all Sorghum mashes were completely liquified.

Furthermore, they showed no retrogradation upon cooling from holding temperature 80 °C to final temperature 65 °C and resting there for 5 minutes, which is indicated by the negative set-back viscosities (see Table 5).

### Example 4. Effect of Different Holding Temperatures

The impact of typical holding temperatures (range from 80-90 °C) on liquefaction performance of ENZ 1 was evaluated.

As shown in Table 6, the performance of ENZ 1 was not dependent on the type of Sorghum, but with increasing holding temperatures, decreasing areas underneath the viscosity curves could be observed for all applied types. On the other hand, the minimum final viscosities were obtained when holding temperature was set to 87.5 °C.

A slight set-back of 34 and 45 cP was measured for two out of the three Sorghums when holding temperature was 87.5 °C. A set-back between 155-186 cP was measured for all Sorghums when holding temperature was 90 °C. Taken together this data indicates that the optimal temperatures for liquefying Sorghum with acid alpha-amylase ENZ 1 are between 85-87 °C.

TABLE 6
Effects of different holding temperatures -

| Sorghum | | 80 °C | 82.5 °C | 85 °C | 87.5 °C | 90 °C |
|---|---|---|---|---|---|---|
| SS-2020-0012 | AUC* | 75323 | 69378 | 66586 | 61728 | 57728 |
| | (Area) | | | | | |
| | FV (cP) | 708 | 558 | 505 | 451 | 519 |
| | SB (cP) | −151 | −99 | −42 | 34 | 163 |
| SS-2020-0021 | AUC | 102858 | 84348 | 81256 | 76326 | 71246 |
| | (Area) | | | | | |
| | FV (cP) | 942 | 620 | 566 | 514 | 606 |
| | SB (cP) | −257 | −183 | −105 | −13 | 155 |
| SS-2020-00023 | AUC | 84194 | 74326 | 70805 | 66999 | 62406 |
| | (Area) | | | | | |
| | FV (cP) | 569 | 518 | 458 | 380 | 451 |
| | SB (cP) | −108 | −60 | −32 | 45 | 186 |

*AUC = Viscosity, Area under viscosity curve;
FV = Final viscosity;
SB = Set back

1. A method of producing a brewer's wort comprising the steps:
a) providing a mash from a grist comprising unmalted Sorghum;
b) adding an alpha-amylase to the mash;
c) heating the mash to the pasting temperature of the unmalted Sorghum starch, and
d) producing a wort,
wherein the alpha amylase has a calculated pI in the range from 4.0 to 4.6.

2. The method according to claim 1 wherein the grist comprises at least 20% (w/w) unmalted Sorghum.

3. The method according to claim 1 wherein the grist consists of unmalted Sorghum.

4. The method according to claim 1, wherein the unmalted Sorghum comprises Sorghum with content of condensed tannins in the range of from 0.2 to 90 mg catechin equiv/g Sorghum.

5. The method according to claim 1, wherein the alpha amylase is selected from the group comprising SEQ ID NO:1, SEQ ID NO:5 and SEQ ID NO:9 and molecules having 60% sequence identity to them.

6. The method according to claim 5, wherein the alpha-amylase has at least 60% identity to the sequence shown in SEQ ID NO: 1.

7. The method according to claim 5 wherein the alpha-amylase has at least 60% identity to the sequence shown in SEQ ID NO: 5.

8. The method according to claim 5, wherein the alpha-amylase has at least 60% identity to the sequence shown in SEQ ID NO:9.

9. The method according to claim 1, further comprising adding a further alpha amylase.

10. The method according to claim 1, further comprising adding a glucoamylase.

11. (canceled)

12. The brewer's wort produced by the method according to claim 1, comprising an alpha amylase with a calculated pI in the range from 4.0 to 4.6.

13. A beer produced by the method according to claim 1.


SEQUENCE LISTING (12 sequences)

SEQ ID NO: 1 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 2 - protein, 486 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVS

SEQ ID NO: 3 - protein, 616 aa, Geobacillus stearothermophilus
AEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRTTTTTTAATSTSKATTSSSSSSAAATTSSSCTATSTTLPITFEELVTTTYGEEVYLSGSISQLGEWDTSDAVKLSADDYTSSNPEWSVTVSLPVGTTFEYKFIKVDEGGSVTWESDPNREYTVPECGNGSGETVVDTWR

SEQ ID NO: 4 - protein, 483 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRYQETSGEYNIQAWTGFNFPGRGTTYSNWKWQWFHFDGTDWDQSRSLSRIFKFHGKAWDWPVSSENGNYDYLMYADYDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGAYDMRNILNNTLVASNPTKAVTLVENHDTQPGQSLESTVQPWFKPLAYAFILTRSGGYPAVFYGDMYGTKGTTTYEIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 5 - protein, 457 aa, Thamnidium elegans
VPLSVLDKRNGVTTLSKRAAAADWKSRSIYQLVTDRFGRSDGSTSACGDLSNYCGGDYKGIQNQLDYIAGMGFDAIWISPIPENTDGGYHGYWAKDFEKLNTNFGSADDLKALVTAAHGKGMYVMLDVVANHAGPASGGDYSGFTFSSASNYHPQCTIDYDNQTSVEQCWVADDLPDINTEDDTIVSKLHSIVSDWVTTYDFDGIRIDTVKHIRKDFWSGYEEAAGVFATGEVFDGDAAYVGPYQDQLSSLINYPLYYAIRDVFSAGSGFSRISDMLSTIKSNFKDPSVLTTFVDNQDNARFLSVKSDMSLYKNALAFTILTEGIPVVYYGTEQGFKGGDDPKNREVLWTSNYDTSSDLYKFIKIVNNDVRQKSDKTVTLDVDVGTNTYAFTHGKNLIVVNNYGSGSTESVTVKVGDSVADGTKLVDAVSNITATVSGGSITFSLKDGLPALFVPSS

SEQ ID NO: 6 - protein, 616 aa, Aspergillus niger; identical to SEQ ID NO: 3

SEQ ID NO: 7 - protein, 429 aa, Pelomonas saccharophila
DQAGKSPAGVRYHGGDEIILQGFHWNVVREAPYNWYNILRQQASTIAADGFSAIWMPVPWRDFSSWTDGDKSGGGEGYFWHDFNKNGRYGSDAQLRQAAGALGGAGVKVLYDVVPNHMNRFYPDKEINLPAGQRFWRNDCPDPGNGPNDCDDGDRFLGGEADLNTGHPQIYGMFRDEFTNLRSGYGAGGFRFDFVRGYAPERVDSWMSDSADSSFCVGELWKEPSEYPPWDWRNTASWQQIIKDWSDRAKCPVFDFALKERMQNGSVADWKHGLNGNPDPRWREVAVTFVDNHDTGYSPGQNGGQHKWPLQDGLIRQAYAYILTSPGTPVVYWPHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 8 - protein, 532 aa, Lactobacillus amylovorus
DTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVP

SEQ ID NO: 9 - protein, 440 aa, Lactobacillus amylovorus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSISDWTHGNTQISNWSDREDVTQNSLLGFYDWNTQNSQVQTYLKNHLERLISDGASGFRYDAATHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNSSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDEFTVKNGYLTGTIQGREVVVLYGDP

SEQ ID NO: 10 - protein, 442 aa, Cordyceps farinosa
ADTNSWKSRTIYFALTDRVARSASDNGGDGCGQLQDYCGGTFKGLEGKLDYIKGMGFDAIWITPVVQNSARGYHGYWASNLYATNSHYGTSDELKGLVNAAHGKGIYIMVDVVANHVGNGPLNEMQPAPLNQGSSYHPACGINYNDQHSIETCRVASDLPDLDTTDPKIRTLYKDWIKWLMSTYKFDGVRIDTVKHVEKDFWPDFAWASGSYTIGEVFSGDPNYVAGYSKLMGGLLNYPVYFPLNRFYQQQNSSQALVDMHNQIGSLVPDPTTLGTFLDNHDNPRFLSQKNDVSLFKNALTYVLLARGIPIVYYGSEQAYAGGGDPQNREDLWRSRFNTNSDMYKFFQALGGVRKSHGGLPGNDHVHLFVESDAYAWSRQDGAVMALTSNIGKGQQRQFCFFTQKNNKTWRGIFDGKTYTSGGDGKLCATVNNGEPIVFVAQ

SEQ ID NO: 11 - protein, 452 aa, Anoxybacillus kamchatkensis
KTERTWQDERIYFIMVDRFNNGNPKNDYDVNVHDPKAYHGGDLQGIIDKLDYIKGMGFTAIWLTPIFANEKGGYHGYWIEDFYKVEEHFGTLDDFKRLVKEAHKRDMKVILDFVVNHTGYNHPWLNDPAKKDWFHEKKDIFNWANQQEVENGWLFGLPDLAQENPEVKAYLFDVAKWWIQETDVDGYRLDTVKHVPKSFWDEFSKEVKSVKQDFFLLGEVWHDDPRYVAEYGKHGIDALIDFPFYKEASTIFSNVDQSLEPLYNVWKRNVAFYDRPYLLGTFLDNHDTVRFTRLALQNRINPVTRLKLGLTYLFAAPGIPIMYYGTEIALDGGEDPDNRRLMNFRTDKELVDYVTKLGELREKLPSLRRGDFELLYEKDGMALFKRTYEKETTVIAINNTSKTQKVTLDDELEQGKELRGLLEGDLVRSKDGKYDIILDRETAEIYVLAPKT

SEQ ID NO: 12 - protein, 566 aa, Valsaria rubricosa
ASNSDWRSRNIYFALTDRVANPSTTTACSDLSNYCGGTWSGLSSKLDYIQGMGFDSIWITPVVENCDGGYHGYWAKALYNVNTNYGSADDLKNFVAAAHAKGMYVMVDVVANHMGSCGIANLSPPPLNEQSSYHTQCDIDYSSQSSIETCWISGLPDLDTTDSTIRSLFQTWVHGLVSNYSFDGLRVDTVKHVEKDYWPGFVSAAGTYAIGEVFSGDTSYVAGYQSVMPGLLNYPIYYPLIRVFAQGASFTDLVNNHDTVGSTFSDPTLLGNFIDNHDNPRFLSYTSDHALLKNALAYVILARGIPIVYYGTEQGYSGSSDPANREDLWRSGYSTTGDIYTTIAALSAARTAAGGLAGNDHVHLYTTDNAYAWSRASGKLIVVTSNRGSSDSSTICFSTQQASGTTWTSTITGNSYTADSNGQICVQLSSGGPEALVVSTATGTATATTLSTTTKTSTSTASCAATVAVTFNELVTTNYGDTIRLTGSISQLSSWSATSGLALSASAYTSSNPLWSVTVSLPAGTSFEYKFVRITSDGTVTWESDPNRSYTVPTCASTATISNTWR